(19) United States
(12) Patent Application Publication
(10) Pub. No.: US 2015/0250456 A1
(43) Pub. Date: Sep. 10, 2015

(54) URINE COLLECTING AND ANALYZING APPARATUS

(71) Applicant: ARGERON MEDIKAL ARASTIRMA SANAYI VE TICARET ANONIM SIRKETI, Muratpasa Antalya (TR)

(72) Inventor: Abdullah Olgun, Antalya (TR)

(73) Assignee: ARGERON MEDIKAL ARASTIRMA SANAYI VE TICARET ANONIM SIRKETI, Muratpasa Antalya (TR)

(21) Appl. No.: 14/430,076

(22) PCT Filed: Jun. 25, 2013
(86) PCT No.: PCT/IB2013/055212

§ 371 Date: Mar. 20, 2015

(30) Foreign Application Priority Data
Jun. 27, 2012 (TR) 2012/07505

(51) Int. Cl. A61B 10/00; B01L 3/00

(52) U.S. Cl. CPC A61B 10/007; B01L 3/5021; B01L 2300/043; B01L 2300/06; B01L 2300/0861

(57) ABSTRACT

A device which provides the functions of a urine collection container, urine centrifugal tube and pipette, used traditionally for urine analysis in a single ergonomic product.

This invention is related to an apparatus to conduct medical urine collection and analysis procedures in a single container. More specifically, this invention is related to an apparatus to collect urine specimen from humans as well as conducting the analysis of the sample received in the one and the same container.

### PRIOR ART

At present, such apparatus used in collecting urine from humans and analyzing the same is generally in the form of container consisting of separate containers. It is described under reference number CN1021 25445 (A), an example of a container where both collecting of urine as well as analysis of the same is conducted and realized by making use of the same container. Here, the urine collector consists of a main body, a pipette, and a centrifugal tube. The main body and the pipette are formed as a single component and such pipette is placed inside the centrifugal tube. During use, the main body and the centrifugal tube are separated, whenever urine of any individual is to be collected, the pipette is placed inside the centrifugal tube, the person involved excretes urine into the main body, and the laboratory technician takes the centrifugal tube and places the same into the test tube where analysis shall be conducted. Such part of the main body dedicated to collecting urine, is removed with the aid of a forceps and then urine analysis is conducted.

However, the disadvantage with respect to the foregoing container is that it requires external intervention while collecting urine from any individual and testing the same and thereafter encountering problems like tilting over and spilling during removing the outer part of the tube with the aid of a forceps. Further, cost and labor are increased as the apparatus comprises of more than one component.

In addition, in another prior art, WO2004 14556 (A1) a supplement is placed inside the collecting tube, and such supplement is accessible from the open end. Obtaining sediment from urine is provided by means of such apparatus; however, it is not possible to conduct any analysis by using the same apparatus. In the prior art, in order to receive urine specimen from women with ease, there are available apparatus enabling collection of urine without spilling, such as a cone-formed cap like extra apparatus as is the case in U.S. Pat. No. 7,998,126. However, these are solely enabling collection of urine; analysis procedure cannot possibly carried out on the same apparatus. And the cost of such an apparatus is expensive due to the material and labor incorporated.

In the prior art document U.S. Pat. No. 4,393,881 discloses a urine collection device for obtaining a urine sample without contamination of either testing personnel or of an evacuated container into which the urine is transferred.

In the prior art document GB2162312 discloses a sterile midstream urine collector having an open-ended tubular body and a cup mounted therein, the cup having its open top equipped with a cover that is formed at least in part of a water-soluble material.

In the prior art document U.S. Pat. No. 4,106,490 a device is described to enable the direct hygienic collection of a urine sample in a specimen tube. The collection aid is formed with a hollow body. A main opening of the hollow body is oriented to receive a female or male voiding stream which is confined in a chamber having specimen and excess urine outlet ports.

The containers used at present are of covered plane cylindrical form, generally of 100 mL volume and are semi-transparent. The cover is not attached to the container, and therefore problems are encountered while using the same, such as the cover falling down, getting contaminated, getting lost, and being forced to close the cover with both hands. Whenever the urine collected in the container is delivered to the laboratory to be duly analyzed, initially organoleptic (color and appearance visually, odor) physical inspection thereof is made by the laboratory technician. Thereafter, a test strip is dipped for chemical examinations, and if required, additional tests are conducted. Yet another stage is microscopic inspection, and in this inspection, in order to facilitate the observation of the elements with specific forms contained in the urine (cells, cylinders, crystals and the like), the urine is transferred from the collection container into the centrifugal tube in order to concentrate the same through centrifugation. Following the centrifugation process, such 9/10 portion remaining in the upper part of the tube is discarded, the remaining 1/10 portion at the bottom is stirred once more, some quantity there from is received with the help of a pipette, a drop from the pipette is placed on the microscope slide and is examined under microscope. However, in the course of realization of the aforesaid procedure, while urine is transferred to the tube, some part of the urine might get spilled around. As such transfer is made practically; the quantity received might possibly be either less or more than an accurate volume of 10 mL, and since the stopper of the tube is not closed in the course of centrifuging, urine droplets (aerosols) are diffused to the ambient air depending on the centrifuging process. And this might contaminate the ambient air. Following centrifuging, it is disposed of not such 1/10 portion at the bottom of the tube, but urine on top is discarded so as to leave a much less quantity of volume or only pellet (precipitate). Then, a drop is taken from this remaining portion, placed on a microscope slide, and examined. Such drop placed on the microscope slide is sometimes more than necessary and sometimes in excess than the quantity sufficient for conducting the procedure in an efficient manner, and causes either the whole bottom surface of the coverglass placed on the microscope slide not fully covered, or outflow from the edges since is more than adequate. And this encumbers the analysis for the person conducting microscopic examinations. Whereas in case of using a pipette (Pasteur pipette or automatic pipette tip) instead of pouring the contents of the tube by rotating the same, there is unnecessary pipette consumption. Concentration of urine at a rate in excess of 1/10 results in the elements with specific forms within the urine are reported more than actually available (false positive result), and thus give rise to unnecessary treatments to be made.

In order to overcome above mentioned disadvantages, according to this invention a compact single component urine collection and analysis apparatus is proposed. Such apparatus that constitutes subject matter of the said invention of ergonomic design, enabling collection of urine inside the container in a clean and hygienic manner particularly in respect of women, is providing for both men as well as women, collection of urine with the same extent of ease and convenience.

More specifically, the object of this invention is to realize the functions required for urine analysis, such as “urine collection container”, “urine centrifugal tube” and “pipette” (Pasteur pipette or automatic pipette tip) all under one single apparatus, facilitating urine collection and analysis procedures for both the person providing urine as well as the conducting analysis, such analysis be made in accordance with scientific criteria, and eliminating any influence of the user on the foregoing.

Such apparatus according to this invention shall be described in detail herebelow by referring to the attached drawings:

In the drawings;

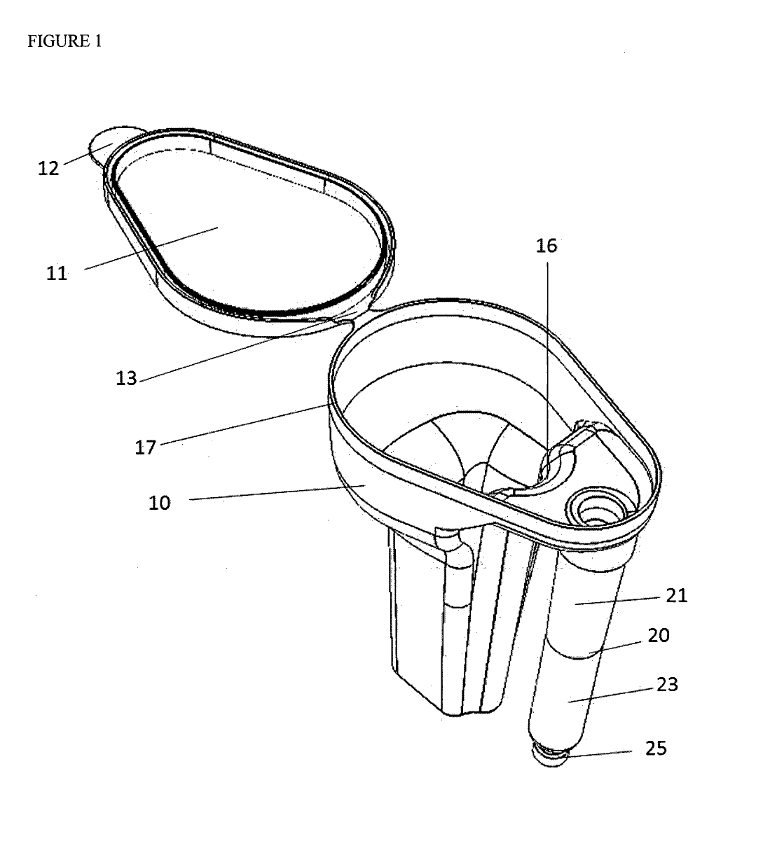

FIG. 1 is the top perspective view of the apparatus.

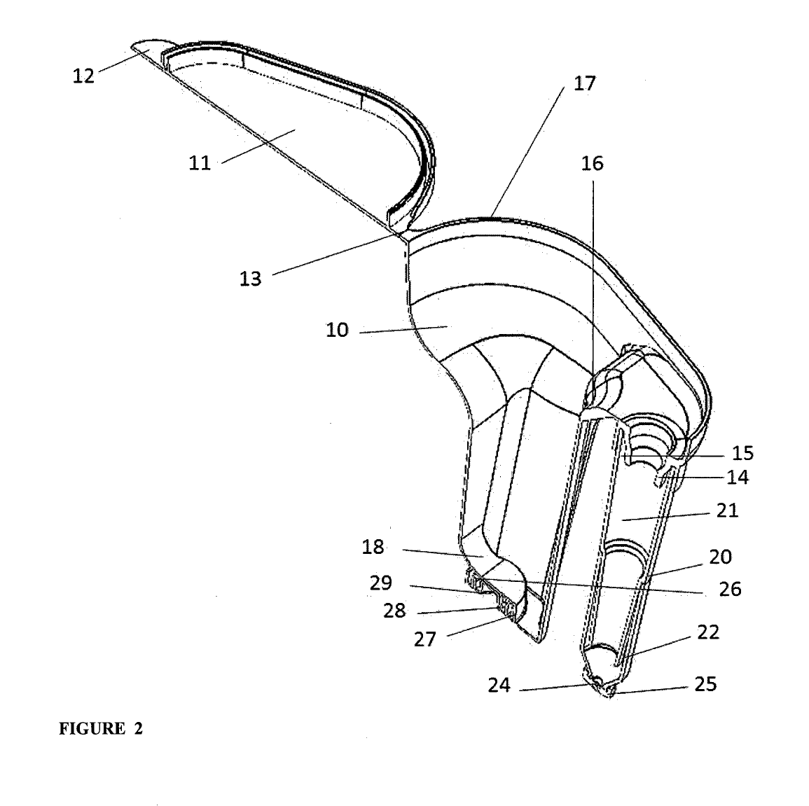

FIG. 2 is the perspective view of the partial section of the apparatus.

Part numbers used on the figures are as follows:
10 reservoir
11 reservoir cover
12 reservoir cover holder
13 reservoir cover-reservoir connection
14 reservoir-tube connection (upper cup-like inkpot)
15 upper cup cavity (like inkpot cavity)
16 reservoir duct
17 reservoir orifice
18 tube cover placement slot
20 urine centrifugal tube
21 upper part of tube (lower cup-like inkpot)
22 lower cup cavity (like ink pot cavity)
23 lower part of tube
24 upper part of tube hole
25 tube bottom stopper
26 tube cover
27 tube cover tube recess
28 tube cover chamber recess
29 tube cover injector needle in-feed well

### BRIEF EXPLANATION OF THE INVENTION

By means of the apparatus that is subject matter of the invention, it has been provided for that the entire functions of urine collection container, urine centrifugal tube and pipette (Pasteur pipette or automatic pipette tip) are realized in the most ergonomic manner under one single product, and standardization according to scientific criteria of such stages of the microscopic analysis process of urine up till visual inspection under the microscope.

In particular, the apparatus that is subject matter of the invention contains at least one urine collection reservoir (10) with a cover (11), at least one urine centrifugal tube (20), at least one tube cover (26), and at least one tube hole stopper (25).

Urine directly provided by the user is easily transferred to said reservoir (10) having a cover (11), by means of the cover (11) of the reservoir (10) getting closed and tilting of the same towards the tube (20). Such tube (20) in possession of a system that comprises of a double cup, one consisting of the upper cup part (14) located at the connection point of the tube and the reservoir, and other located at the lower part (21) of the tube, enables that for centrifuging full 10 mL urine is duly kept, and following the centrifuging process, exactly 1 mL of urine remains at the lower part of the tube. From the hole (24) that is provided at the bottom of the tube (20) a drop of urine of volume 20 microL is transferred onto the microscope slide of the microscope. The diameter of such hole (24) is preferably 0.95 mm. in order to provide predetermined volume.

### DETAILED DESCRIPTION OF THE INVENTION

In order to achieve the objects of the invention indicated hereabove and to realize several other advantages, such apparatus described in detail herebelow has been developed. The apparatus contains at least one cover (11) enabling the reservoir (10) to seal in a hermetic manner after urine is placed therein, yet closed and opened with ease by using only one hand, such tube (20)-reservoir (10) connection (14) serving at the same time as an upper cup, a urine collection reservoir (10); upper part of the tube (21) that is connected to the reservoir the lower part of which is at the same time the lower cup, lower part of the tube (23) and a urine centrifugal tube (20) that consists of the tube bottom stopper (25); a tube cover (26) to close the upper orifice of the centrifugal tube (21) when the centrifugal tube (20) is removed for inspecting from the connection point of the tube-reservoir, that is from the upper cup (14), as well as a tube bottom stopper for closing in a hermetic manner the open end (24) of the lower part of the urine centrifugal tube (23). The tube cover (26) is designed at the same time so as to close the tube-reservoir connection (14).

The orifice part (17) of said reservoir (10) incorporates such a form that both convenience and hygiene is provided, regardless of whether or not the individual supplying urine is a man or a woman. Such form might possibly be of a pear-shaped geometry. The upper orifice opening (17) of the reservoir (10) is formed to enable particularly women fouling their hands and spilling around. The opening orifice of the reservoir is wide, with diameter reduced towards the lower part, and in order to avoid any spillages while getting narrow, recesses and steps are incorporated. A cover (11) is provided at the opening orifice of the reservoir (10) in such a manner that it exactly fits to said orifice and avoids spilling and leaking out of the urine placed inside the reservoir (10) hermetically. The cover (11) being constructed preferably as a single element together with the reservoir (10), eliminates the difficulty of removing and placing somewhere else the cover (11) while providing urine and then finding and attaching the same thereafter. The cover (11) enables to hold the reservoir (10) externally without placing hand into the closet. Thereby, it is avoided that the hand gets in contact with the closet and is fouled. The connection of the cover (11) with the reservoir (10) might be provided by means of a hinge mechanism that is constructed and formed by reducing thickness of the material enabling opening and closing the same several times, as well as in a detachable manner. The holding part (12) on the cover (11) enables to hold the apparatus with only by the thumb and index finger in such a way that the loss of sterilization is avoided in the inner part of the cover (12). The internal structure of the reservoir (10) is in the form of a duct (16) that is slanted so as to avoid spilling outwards during collection of urine and incorporating a stepped or graded form to enable transfer of urine within the reservoir (10) by means of bending of the reservoir (10) towards the tube-chamber connection (14). The orifice of the reservoir (10) being wider than the lower base part, is providing convenience in the course of urine collection. As the shape of the narrow reservoir (10) downwardly the outer surface of the reservoir (11) will not get into contact with the inner part of the closet during collection of urine. In addition, to ensure that the reservoir stands straight after collecting urine, the bottom surface of the base is plane. The volume of the said reservoir (10) up to the orifice of the tube ranges between 100 to 150 mL and is preferably about 130 mL. Whereas the height of the reservoir is such that the entire strip is used in the chemical examination of urine, without bending the same.

On the bottom part of the reservoir standing on its base, is included a cover (26) insertion slot (18) for placement of the tube cover (26) covering the tube (20)-reservoir (10) connection (14) as well when the tube (20) is detached and removed from the reservoir, while the centrifugal tube (20) is connected to the chamber (10). Said cover slot is so designed that the cover (26) is removed and installed with ease. When the cover (26) is not used, it is inserted into such slot (18) that is provided under the reservoir (10). For cases where it is desired that the reservoir-tube connection (14) and tube (20) are to be closed at the same time, the apparatus might possibly be manufactured with two tube covers (26) and two tube slots (18).

The urine centrifugal tube (20) contains a tube upper part (lower cup) (21), a tube lower part (23), a tube bottom stopper (25) and a tube cover (26). The tube is formed separately as lower (23) and upper (21) parts for formation of the cup (22) since this is required by molding technique; however if allowed by the molding technique, it might as well be produced as a single item. The bottom stopper (25) of the said tube (20) is provided with the purpose of closing the hole (24) under the tube (20), and if so desired, such stopper (25) might be formed as a single element together with the lower part of the tube (23) and provided in such a form that is opened and closed. The connection of said reservoir (10) and said urine centrifugal tube (20) is constructed in the form of an upper cup (14) and so as to enable removal and placement of the centrifugal tube to and from the reservoir (10), and the lower cup part (21) of the said centrifugal tube consists of a tube lower part (23) with at least one hole (24) and a tube bottom stopper (25) closing said hole (24).

After the person individual providing urine specimen fills the urine into the reservoir (10), he/she closes the cover (11) and delivers the same to the technician who would be carrying out the analysis. The technician to conduct and carry out the analysis, without opening the cover (11) at all, tilts the apparatus towards the side where the tube is located, and ensures that the urine inside the reservoir is duly transferred into the tube (20). When the tube (20) is filled, the apparatus is placed onto the horizontal surface and thereby such portion of the urine inside the tube (20) which is not required for the analysis, returns back to the reservoir (10). By means of the air cavity (15) generated by the said tube (20) and cup (21) upper orifice at the connection point and surface of the tube and reservoir connection, of total volume 1 mL, it is ensured that exactly 10 mL urine remains inside the tube (20). Thus, it is ensured that a constant volume of urine is transferred into the tube (20) without requirement for any further measurement or adjustment. Thereafter, as and when the tube (20) is detached from the reservoir (10), a cavity of 1 mL is generated on the upper part of the tube (20), allowing for the closing of the cover (26). Since the cover of the tube is closed, no urine droplets suspended in the ambient air is present in the course of the centrifuging process. Following the centrifuging process, when the tube cover (26) is opened and the urine inside the tube is poured, by means of such cavity (22) of volume 1 mL generated at the orifice of the lower cup part of the tube, there remains exactly 1 mL of urine at the bottom of the tube. If so desired, such cavity might as well be designed as 0.5 mL in order to concentrate urine 20 times. Therefore, elements of specific pattern (cells, crystals, cylinders, and such similar) contained in the urine and which are to be examined under microscope are 10 times concentrated as 9/10 parts of the liquid portion of urine is disposed of.

Urine remaining inside the lower part of the tube (23) is stirred once again, the stopper of the hole (25) at the bottom is removed, and one drop of urine is dropped onto the microscope slide through gravity while holding the tube in an upright position. It is not further required to use a pipette for this operation. Since the bottom hole (24) diameter of the tube (20) is preferably 0.95 mm, one drop of urine flowing out when the tube is at upright position is of 20 microL volume.

To allow for the drop to fall down with ease, and to avoid any increase in its volume through contact with the outer edges, the hole (24) is extended in the form of a duct. And such volume is as much as the quantity recommended for urine microscopy.

Tube cover (26) is designed to cover tube-chamber connection (14) at the same time. If the tube (20) is not to be installed into the reservoir (10) any more, and the urine inside the reservoir (10) is to be kept for using the same in the next analysis, the tube (20) is disposed of and connection (14) between cover (26) and tube is closed. If desired, collection of urine into the reservoir (10) might as well be carried out without any tube (20) while reservoir-tube connection (14) is closed with the tube cover (26). In such a case, the tube (20) might be installed into the tube by the technician prior to the analysis. If it is so desired that the tube (20) and reservoir-tube connection (14) are closed at the same time, the tube cover (26) might be supplied as two units.

Urine centrifugal tube (20) is of suitable dimensions in order to centrifuge the urine for any microscopic analysis; it comprises of four parts, namely the upper part (21), the lower part (23), bottom hole stopper (25) and tube cover (26). Said parts might both be formed as an integral single part, as well as discrete independent parts, depending on the manufacturing technique.

Notwithstanding the foregoing or if the technique is applied in a different manner, it might consist of more or less components upon maintaining the internal features. Thanks to the air cavity (22) of volume 1 mL between the part of tube upper part (21) with diameter reduced downwards with an angle of 2° and external surface (23) of the tube, a lower cup (21) feature has been attained. Following the centrifuging process, when the cover of the tube (26) is opened and urine inside the tube is poured out, thanks to the cavity (22) of volume 1 mL at the lower cup (21) system of the tube, exactly 1 mL of urine remains at the bottom of the tube (20). Therefore, elements of specific pattern (cells, crystals, cylinders, and such similar) contained in the urine and which are to be examined under microscope are 10 times concentrated as 9/10 parts of the liquid portion of urine is disposed of, without requirement for use of any other material to that effect. Thanks to the total volume of the surface of such connection area (14) such air cavity (upper cup) (15) generated by narrowing of the diameter downwards of the connection part of the reservoir (10) with the tube and where no urine can possibly infiltrate between the same and external surface (21) of the tube, and part of the reservoir (10) extending into the tube (20), when the tube (20) is detached from the reservoir (10) for centrifuging, it is ensured that the volume of urine inside the tube prior to the centrifuging process is exactly 10 mL. Following centrifuging, while the entire urine of 9 mL at the upper part of the tube (20) is poured and disposed of, thanks to the urine of 1 mL remaining at the bottom of the tube (23) filling into the cavity of volume 1 mL (second cup) (22) remaining between the outer surface of the tube as a result of the diameter of the tube upper part (21) getting narrower downwards, and stays there, 1 mL of urine remains for the microscopic inspection. In order to enable that the remaining urine of 1 mL is placed on the microscope slide for inspection, following re-stirring of the same after centrifuging, on the base of the conical shaped part (23) of the tube, a hole (24) of sufficient width so as to provide for the exit of exactly 20 microL of urine in a drop, of diameter ranging from 1 to 2 mm, and preferably 0.95 millimeters, extended in the form of a duct, which in turn is closed with a stopper (25). Such stopper (25) is so designed that it would not get removed during centrifuging, yet of such tightness to close the said hole (24) that is possibly opened manually. Thanks to the cavity (22) provided in the tube (20) between the upper part (21) of the tube and lower part (23) of the tube, even when the tube (20) is turned fully upside down, it is prevented the outpouring of such 1 mL of urine at the bottom, while the tube (20) is in an inverted position, the stopper (25) is removed and then the tube is reverted, thus 20 microL of urine is possibly transferred onto the microscope slide with the aid of gravity. Therefore, there is no requirement for using any Pasteur pipettes or pipette tips for transferring the urine inside the tube (20), and insertion of any further element into the urine. Volume of the referred to cavity (22) ranges between 0.50 mL to 2 mL, and is preferably 1 mL.

Tube cover (26) is being used for closing both the tube (20) as well as the connection part (14) of the reservoir (20) with the tube (10), thanks to its two recesses, one plugging the orifice part (27) of the tube, while the other plugging the connection part (28) of the chamber with the tube.

While the tube cover (26) is in its slot (18) on the chamber (10), sterile urine might possibly be taken upon entering the reservoir (10) by means of an injector through the tube cover injector needle well (29) at the center. A line is marked on the outer side of the upper part (21) of the tube to indicate the level of volume therein is 10 mL when the tube (20) is detached from the reservoir (10) and is standing upright. The tube (20) might also be used independent of the reservoir (10), and 10 mL of urine might be placed therein without requiring any further measurement procedure.

1. A urine collection and analysis apparatus that consists of at least one urine collecting reservoir (10) within which the urine is filled and at least one urine centrifugal tube (20)—that has 2 air cavities [upper (15) and lower (22); upper air cavity (14) allowing to keep exactly a desired/calibrated volume of urine inside the tube and lower air cavity (22) allowing to keep exactly a desired volume of concentrated urine at the bottom of the tube (20) after decanting upper part of centrifuged urine] and bottom hole (24) that allows to transfer—by just gently squeezing the tube (20)—of a desired volume of concentrated urine on microscope slide or unconcentrated sterile urine for culture—detachably connected thereto in such a manner that it is attachable as well as removable there from wherein said tube (20) is located on a side of the reservoir (10) so as to provide that the urine inside the reservoir (10) to be duly transferred into the tube (20) when the apparatus is tilted towards the said side and after the tube (20) is filled with urine the reservoir (10) is tilted toward its original vertical position and thanks to the upper air cavity (15) excess urine flows back to the reservoir (10) and exactly a desired volume of urine remains inside the tube (10).

2. Apparatus according to claim 1, where the said urine collecting reservoir (10) upper orifice opening is of such geometry that is convenient for collecting urine from men and women.

3. Apparatus according to claim 1, where the upper opening of the said reservoir (10) contains at least one cover (11).

4. Apparatus according to claim 2, where the section of the upper orifice opening of the said reservoir (10) is in the form of a pear.

5. Apparatus according to claim 3, where the said cover (11) is formed detachably or formed as an integral element with the reservoir (10).

6. Apparatus according to claim 5, where a holding part (12) is provided on the said cover (11).

7. Apparatus according to claim 1, where the connection between said reservoir (10) and said urine centrifugal tube (20) is constructed in the form of an upper cup (14), enabling attachment as well as detachment of the centrifugal tube to and from the reservoir (10), where the lower cup part (21) of the said centrifugal tube contains a tube lower part (23) with at least one hole (24), and a tube bottom stopper (25) covering the said hole (24).

8. Apparatus according to claim 7, where the said tube upper part (lower cup) (21), tube lower part (23) and tube bottom stopper (25) are constructed as separate components.

9. Apparatus according to claim 7, where said tube upper part (lower cup) (21), tube lower part (23) and tube bottom stopper (25) are formed as an integral part.

10. Apparatus according to claim 7, where at the upper orifice (15) of the said lower cup part (21), a cavity of volume about 1 mL or 0.5 mL.

11. Apparatus according to claim 7, where it includes a tube cover (26) providing to close the upper orifice of the centrifugal tube (21) whenever the said urine centrifugal tube (20) is detached from the upper cup (14).

12. Apparatus according to claim 11, where said cover is provided more than one in number to close the chamber-tube connection (14) as well.

13. Apparatus according to claim 1, where the orifice part of the said reservoir (10) is wide, and gets narrower downwardly by providing recesses and thanks to such narrowing, the contact of the outer surface with the inner surface of the closet while collecting urine is prevented.

14. Apparatus according to claim 3, where the narrowing part of the said reservoir (10) is provided in the form of a duct to ensure that urine is transferred into the centrifugal tube with ease and in a predetermined amount.

15. Apparatus according to claim 1, where the internal volume of the said reservoir ranges approximately between 100 mL to 150 mL.

16. Apparatus according to claim 1, where the internal volume of the said reservoir is approximately 130 mL.

17. Apparatus according to claim 1, where whenever the said centrifugal tube (20) is detached from the said reservoir (10), it is supplied a cover placement slot (18) on the base part of the said reservoir (10) for placement of the said cover (26) when the centrifugal tube (20) is attached to the chamber (20), in order to close the part for connection between chamber and tube.

18. Apparatus according to claim 7, where the length of the said tube hole (24) is between 0.5 to 3 mm.

19. Apparatus according to claim 7, where the diameter of the said tube hole (24) is 0.95 mm.

20. Apparatus according to claim 7, where the said hole (24) is extended in the form of a duct.

21. Apparatus according to claim 7, where the diameter of the said tube upper part (21) is reduced as moved downwardly.

22. Apparatus according to claim 7, characterized by providing of a cavity (22) between the said lower tube part (21) and the tube lower part (23).

23. Apparatus according to claim 7, where the volume of the said cavity (22) ranges from 0.50 mL to 2 mL.

24. Apparatus according to claim 7, where the volume of the said cavity (22) is 1 mL.

25. Apparatus according to claim 10, where a well (29) is provided on the lower outer part of the said tube cover (26), in order to take urine from the reservoir with the help of an injector while attached to the slot (18) inside the reservoir (10).

26. Apparatus according to claim 7, where by means of placing a marking to show the amount as “10 mL” onto the outer side of the tube upper part (21), it is provided that 10 mL of urine is placed therein without using any further measurement when the tube (20) is used independently.
